(19) United States
(12) Patent Application Publication
(10) Pub. No.: US 2013/0236853 A1
(43) Pub. Date: Sep. 12, 2013

(54) 3D DUCTILE AND PERFORATED RETAINING SHEET

(71) Applicants: Robert Axelsson, Granna (SE); Lars Magnus Bjursten, Limhamn (SE); Niklas Johansson, Jonkoping (SE); Erik Lennings, Huskvarna (SE)

(72) Inventors: Robert Axelsson, Granna (SE); Lars Magnus Bjursten, Limhamn (SE); Niklas Johansson, Jonkoping (SE); Erik Lennings, Huskvarna (SE)

(73) Assignee: TIGRAN TECHNOLOGIES AB (PUBL), Malmö (SE)

(21) Appl. No.: 13/699,423

(22) PCT Filed: May 24, 2011
(86) PCT No.: PCT/SE11/50647

§ 371 Date: Feb. 4, 2013

Related U.S. Application Data
(60) Provisional application No. 61/344,163, filed on Jun. 3, 2010.

(30) Foreign Application Priority Data
Jun. 3, 2010 (SE) 1050572-5

(51) Int. Cl. A61C 8/02; A61D 5/00; A61K 6/04

(52) U.S. Cl. CPC A61C 8/0006; A61K 6/04; A61D 5/00
USPC 433/173

(57) ABSTRACT

The present invention describes a sheet 1 intended for retaining a granular material inserted or implanted in a human or animal body, said sheet 1:
being made of a plastic deformable and non-toxic material chosen from the group consisting of a metal, metal alloy, a polymeric material or a non-woven fabric;
comprising a matrix 2 and several holes 3, each hole 3 having a smallest dimension from one side of the hole 3 to another side of the hole 3, through a geometrical centre of the hole 3, of at least 50 μm and of maximum 2 mm;
being stretchable in both a longitudinal 4 and transversal 5 direction of the sheet 1; and
being 3D ductile.

### FIELD OF INVENTION

The present invention relates to a sheet intended for retaining a granular material inserted or implanted in a human or animal body, such as granular material inserted or implanted dentally.

### TECHNICAL BACKGROUND

There are known membranes for retaining a medically inserted granular material. One such membrane is described in U.S. Pat. No. 6,244,868, which discloses a tissue regeneration barrier for root from dental implants. The barrier is said to facilitate osseointegration of implants placed with transmucosal healing elements immediately into tooth extraction sites. The barrier comprises an absorbable circumferential membrane arranged to exclude epithelial cells but not osteoblasts from the tooth extraction socket in which the implant is placed. The membrane may be supplemented by a sheet membrane of titanium mesh or foil of absorbable material such as bovine or porcine collagen or synthetic polymer. Other dental membranes are disclosed in e.g. U.S. Pat. No. 6,030,218 and US 2010/0086890. U.S. Pat. No. 6,030,218 discloses a sub-periosteally implantable prosthesis support structure for a fixed or detachable dental prosthesis. The support structure may be made, partly or wholly, from either non-resorbable material, such as titanium stock or mesh, or from a resorbable material such as Vicryl™. US 2010/0086890 discloses a removable dental positioning appliance which also is made of a metallic mesh material, such as e.g. titanium.

One decisive drawback of the membrane structures disclosed above and also other such known membranes is their incapacity of being shaped in three dimensions without losing other important properties.

In EP 0 654 250 A1 there is disclosed what is said to be a form-fitting mesh implant made of e.g. titanium. The mesh grid is intended for the fixation of bone fragments at bone fracture sites, such as e.g. in a cranium of a human. The mesh of D1 has a plurality of orifice plate sections that accept bone screws and which have connecting arms coupling each orifice plate section. Each such connecting arm has a bend.

One aim of the present invention is to provide a sheet intended for retaining a granular material inserted or implanted in a human or animal body, such as for dental applications, which sheet solves the problem above by being ductile for specific uses without losing any properties.

### SUMMARY OF INVENTION

The purpose above is achieved by a sheet intended for retaining a granular material inserted or implanted in a human or animal body, said sheet:
being made of a plastic deformable and non-toxic material chosen from the group consisting of a metal, metal alloy, a polymeric material or a non-woven fabric;
comprising a matrix and several holes, each hole having a smallest dimension from one side of the hole to another side of the hole, through a geometrical centre of the hole, of at least 50 μm and of maximum 2 mm;
being stretchable in both a longitudinal and transversal direction of the sheet; and
being 3D ductile.

According to one specific embodiment of the present invention, the sheet is perforated and made of a plastic deformable and non-toxic material chosen from the group consisting of a metal, metal alloy, or a polymeric material. The expression “perforated” in this context implies that holes have been provided in the sheet material. A non-woven fabric has holes in itself and does not have to be perforated according to the present invention. Therefore, according to one specific embodiment of the present invention, the sheet is made of a plastic deformable and non-toxic non-woven fabric in which the several holes are inherent in the material of the sheet.

The sheet according to the present invention is intended for retaining a granular material inserted or implanted in a human or animal body, especially for granular material inserted for dental applications, e.g. for retaining porous titanium or titanium alloy granules inserted in a cavity around a dental implant. Such titanium or titanium alloy granules are e.g. inserted to avoid the risk of periimplantitis by promoting bone ingrowth and hence increase the stability and anchoring of the dental implant. The sheet according to the present invention shall fulfill two main purposes. The first thing is to retain the granular material in place, such as e.g. in the cavity where the material is inserted. The second thing is to allow passage of e.g. supplied nutrient solution or other fluids, but also to allow growth of different cells through the sheet. Fifty μm is a reasonable sized hole that is feasible to manufacture using conventional techniques such as etching, laser cutting, water cutting and punching. The maximum of the smallest dimension from one side of one hole to another side of that hole, through a geometrical centre of the hole, is 2 mm.

In view of the size range of the smallest dimension of the holes of the matrix, the sheet according to the present invention is optimized for retaining granular material inserted for dental applications, e.g. for retaining porous titanium or titanium alloy granules inserted in a cavity around a dental implant, such as a titanium screw, to allow for the bone cells of the jaw bone to grow through the granules so that the titanium screw is securely anchored. The size of the holes allow for bone ingrowth, while at the same time ensures that the granular material is retained. This would for instance not be possible with a mesh described in EP 0 654 250 A1, in which description it is obvious that the dimensions of both screw holes and other holes of the mesh have a larger dimension so that granular material would penetrate through the mesh and thus would not be retained.

Another important difference of the sheet according to the present invention and the mesh disclosed in EP 0 654 250 A1 is the existence of specific screw holes. The mesh holes in D1 are so large so that screw holes with a somewhat smaller dimension have been incorporated for the possibility of fixating the mesh with screws. This is not needed with the sheet according to the present invention, where the dimension of the holes are such so that fixating elements, like screws, may be used directly into the matrix holes, for fixation, while at the same time provide for retention of the granular material. One may describe this difference in terms of that the matrix of the sheet according to the present invention defines the holes entirely, i.e. there are no additional screw holes incorporated in the matrix.

Furthermore, the existing screw holes of the mesh in EP 0 654 250 A1 as well as the solution of a bent arm in the connection between the ring material around the screw holes also points away from the sheet according to the present invention. First of all, the screw holes are static parts of the mesh in EP 0 654 250 A1, while the sheet according to the present invention may be said to have no such static parts. Secondly, the bent per se in the connection in EP 0 654 250 A1 ensures for the stretchability. This is a restriction in view of the fact that the matrix may only be stretched in the elongation direction of these bends. The sheet according to the present invention is not directed to having bent arms with a clear bend angle for achieving the stretchability and 3D ductility.

Some of these differences are of course also linked to the different intended usage of the sheet according to the invention in comparison to the mesh of EP 0 654 250 A1.

Furthermore, as the sheet according to the present invention is intended for medical uses, the material of the sheet should be non-toxic. Moreover, the material of the sheet should be plastic deformable so that it is possible to shape the sheet in a maintained shape. However, this is not enough to achieve a 3D ductile sheet. One other very important technical feature of the sheet according to the present invention is the stretchable ability in both longitudinal and transversal direction of the sheet. This feature makes the sheet 3D ductile in a way so that the sheet may be shaped in a desired and preserved way without losing any other properties. This is a feature and ability, which known retaining membranes, such as the ones disclosed in U.S. Pat. No. 6,030,218 and US 2010/0086890, do not have. Such known membranes and other similar membranes, like regular nets or meshes, may have a stretching ability in one direction, i.e. either longitudinal or transversal, but not in both directions. Hence, they are not 3D ductile. The ability of being 3D ductile according to the present invention implies that sheet may be shaped without obtaining a crease. If a thin aluminum foil is shaped, such foil will obtain a crease.

Moreover, the ability of being stretchable in both a longitudinal and transversal direction may be achieved in different ways according to the present invention, which is explained below.

### BRIEF DESCRIPTION OF THE DRAWINGS

FIG. 1 shows a perforated sheet according to the present invention.

FIG. 2 shows an enlarged section from the perforated sheet in FIG. 1, which section is marked in FIG. 1.

FIGS. 3 and 4 show the same perforated sheet, seen form above and from the side, respectively.

FIG. 5 also shows a perforated sheet according to the present invention, which sheet comprises a preformed fixation hole.

FIGS. 6 and 7 show that perforated sheet, seen from above and from the side, respectively.

### SPECIFIC EMBODIMENTS OF THE INVENTION

As mentioned above, the material of the sheet shall be plastic deformable and non-toxic. According to one specific embodiment of the present invention, the plastic deformable and non-toxic material is chosen from the group consisting of stainless steel or an alloy thereof, aluminium or an alloy thereof, niobium or an alloy thereof, zirconium or an alloy thereof, tantalum or an alloy thereof, or titanium or an alloy thereof. The plastic deformable and non-toxic material may also be chosen from a biocompatible polymeric material. Examples of such are PEEK (polyether ether ketone), PC (polycarbonate), polyethylene, polypropene and polyurethane. Other possible examples are biocompatible and biodegradable polymeric materials, such as polylactic acid or polyglycolic acid materials.

Metals and metal alloys are suitable for medical applications in view of the fact that these materials, in particular titanium and titanium alloys, are used in implants, especially in dental implants. According to one specific embodiment of the present invention, the plastic deformable and non-toxic material is chosen from titanium or an alloy thereof.

As described above, the plastic deformable and stretchable feature of the sheet according to the invention is of great importance. All materials could be said to be stretchable in some sense, i.e. if the applied force is large enough. However, this is not what “stretchable” implies in relation to the present invention. The sheet is stretchable if only a very small force is applied, such as the force possible to exert with a thumb. The sheet is intended to be 3D ductile by e.g. a surgeon or dentist before use without the help of any devices. Only the use of hand power should be enough.

Moreover, the feature of being stretchable in both a longitudinal and transversal direction of the sheet is also of great importance. According to the present invention, this may be accomplished in different ways. According to one specific embodiment, the matrix is stretchable in both a longitudinal and transversal direction of the sheet. One example of the embodiment is a matrix comprising rings which are flexibly combined with one another. Every ring is somewhat stretchable creating a stretchable matrix. Another possibility is that the rings are non-stretchable but may be displaced with respect to each other. One such example is in the form of a medieval shielding armour. According to another specific embodiment of the present invention, the holes are stretchable in both a longitudinal and transversal direction of the sheet. According to yet another specific embodiment of the invention, the matrix and the holes are stretchable in both a longitudinal and transversal direction of the sheet. The example shown in the figures is a perforated sheet where both the matrix and the holes are stretchable in both a longitudinal and transversal direction. A non-woven fabric, which may have a structure of a matrix and holes without being industrially perforated, is another example of a material and structure where both the holes and the matrix may be plastic deformable to achieve the desired properties according to the present invention. As is evident from above, a rigid net or mesh is not embodied by the present invention in view of the fact that such net or mesh do not exhibit such a stretchable feature.

Moreover, according to one specific embodiment of the present invention, the sheet is more stretchable in a longitudinal direction than in a transversal direction, or vice versa. The stretchable ability may also vary, both in relation to the longitudinal direction and the transversal direction, at different places of the sheet.

The appearance of the sheet and its matrix and holes may vary according to the invention. According to one specific embodiment, the holes are non-round. According to another embodiment, the holes are curved or angular. According to yet another embodiment of the present invention, the holes create a repetitive pattern on the sheet. As is shown in the figures, such a repetitive pattern of curved holes is provided. It is important to understand that non-repetitive patterns also may be provided according to the present invention, such as patterns built up by a mixture of round, non-round, curved and angular holes, as long as the sheet is stretchable in both a longitudinal and transversal direction of the sheet and is 3D ductile.

As is mentioned above, the sheet according to the present invention finds use in medical applications, such as for retaining granular material inserted e.g. around a dental implant. According to one specific embodiment of the present invention, the sheet also comprises at least one preformed fixation hole. One such fixation hole is e.g. intended to be fixed around a dental crown of an implant. As such the sheet may be fixated more securely for such applications. The fixation hole according to the present invention should not be confused with a screw hole according to EP 0 654 250 A1. If present, the fixation hole is positioned in a defined space of the sheet matrix and not incorporated in the entire structure as in the case of the screw holes in EP 0 654 250 A1. This is also the fact if more than one fixation hole is provided in the sheet matrix of the present invention. However, it is of course important to realize that sheets according to the invention not having any fixation holes are fully possible for use to secure and retain granular material inserted in a cavity, both in the mouth of a patient but also at other possible places in a human or animal body.

The possible sizes of the holes of the sheet are linked to the size of the granules of the material intended to retain. For dental applications, granules having a diameter size of between 0.7 and 1 mm are often used. To retain such granules, often titanium or titanium alloy granules, securely, the holes of the sheet may not be too large. Therefore, according to one specific embodiment, to be optimized for retaining granules having a diameter size of between 0.7 and 1 mm, the holes of the sheet matrix have a smallest dimension from one side of one hole to another side of that hole, through a geometrical centre of that hole, of maximum 0.5 mm. If the granules are smaller, the holes of course have to be smaller. The sizes of the holes may also be larger for other types of medical applications when the granules are larger. Nevertheless, the size of the smallest dimension from one side of one hole to another side of that hole, through a geometrical centre of that hole, may vary within the given range of from 50 μm to 2 mm, in relation to the size of the granules and thus the intended use.

The smallest dimensions of the holes described above are given for a sheet directly after production, i.e. before it has been shaped for its intended use. However, even if the holes are stretchable, the smallest dimensions of the holes given above also apply for a sheet according to the present invention which has been shaped for its intended use.

The perforated sheets according to the present invention may be produced by different techniques, such as e.g. by etching, laser cutting, water cutting, punching, but also other techniques may be possible.

### DETAILED DESCRIPTION OF THE DRAWINGS

In FIG. 1 a perforated sheet 1 intended for retaining a granular material inserted or implanted in a human or animal body is shown. The perforated sheet 1 is built up by a matrix 2 and several holes 3. In this case the holes 3 are curved and the pattern of the holes 3 is repetitive. As the holes 3 are evenly curved, the geometrical centre of such a curved hole 3 will be located in the middle of the curved hole 3 below the bending top of the curved hole 3.

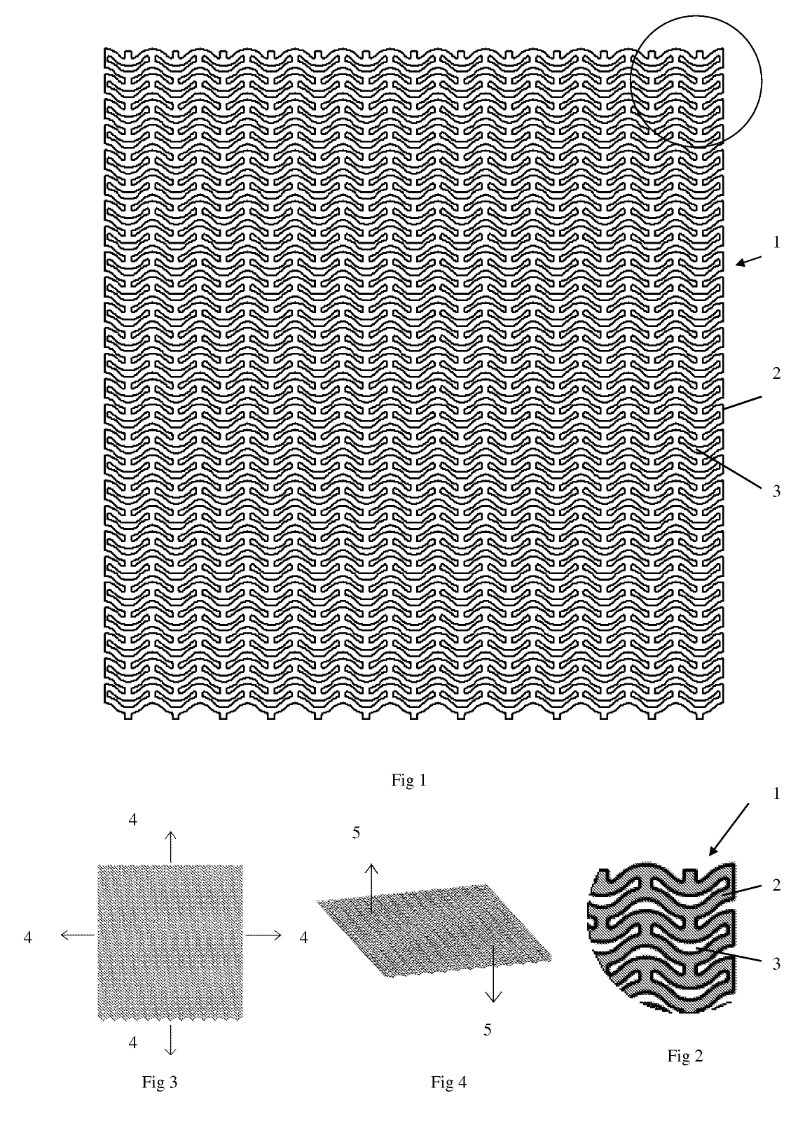

In FIG. 2 an enlarged section of the perforated sheet 1 of FIG. 1 is shown.

In FIG. 3, the perforated sheet 1 of FIG. 1 is shown in scaled-down size, seen from above. The longitudinal 4 directions are marked in FIG. 3, and the directions 4 should be interpreted as all possible directions in the same plane as the perforated sheet 1.

In FIG. 4, the perforated sheet 1 of FIG. 1 is shown in scaled-down size, seen from the side. The transversal 5 directions being directions out from the plane of the sheet 1 are marked in FIG. 4.

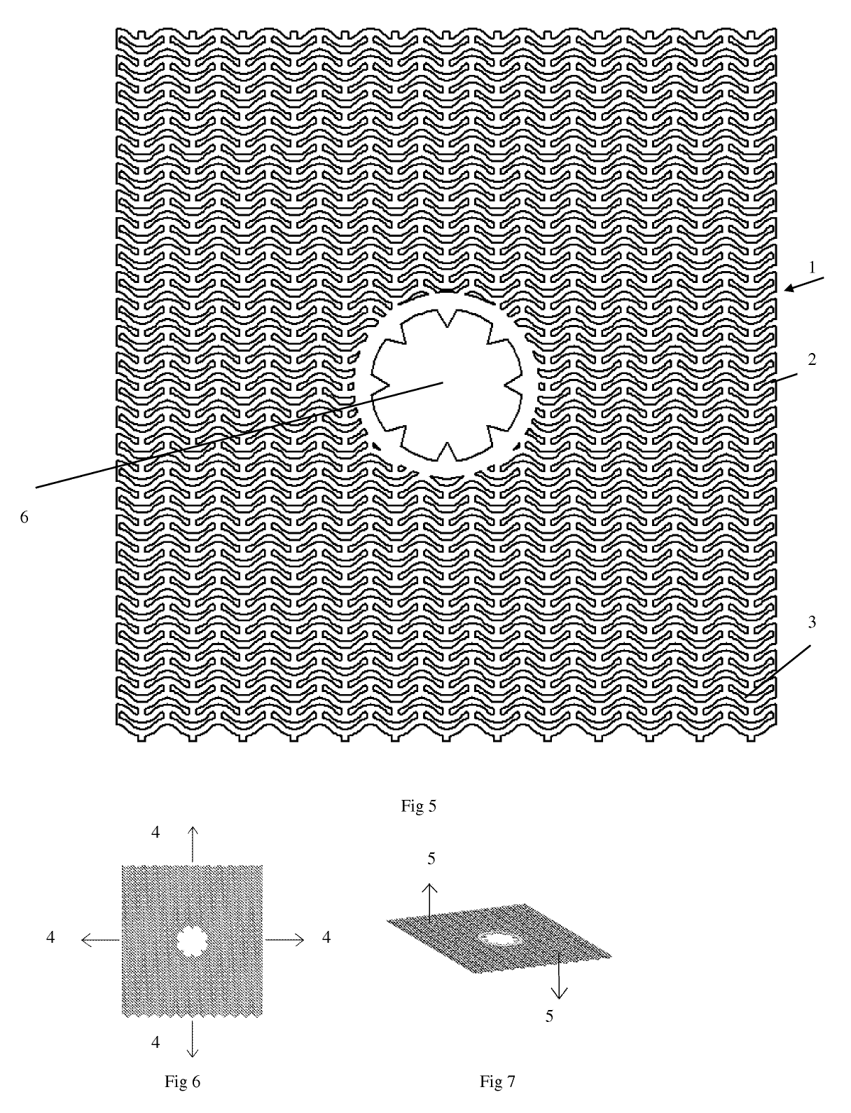

In FIG. 5 there is shown a similar perforated sheet 1 as in FIG. 1, however this perforated sheet 1 comprises a preformed fixation hole 6 intended to be placed around e.g. a dental implant.

FIGS. 6 and 7 are figures in accordance with FIGS. 3 and 4, however for the perforated sheet 1 shown in FIG. 5.

What is claimed is:

1. A sheet intended for retaining a granular material inserted or implanted in a human or animal body, said sheet:
being made of a plastic deformable and non-toxic material chosen from the group consisting of a metal, metal alloy, a polymeric material or a non-woven fabric;
comprising a matrix and several holes, each hole having a smallest dimension from one side of the hole to another side of the hole, through a geometrical center centre-of the hole, of at least 50 μm and of maximum 2 mm;
being stretchable in both a longitudinal and transversal direction of the sheet; and
being 3D ductile.

2. The sheet according to claim 1, wherein the sheet is made of a plastic deformable and non-toxic non-woven fabric in which the several holes are inherent in the material of the sheet.

3. The sheet according to claim 1, wherein the sheet is perforated and made of a plastic deformable and non-toxic material chosen from the group consisting of a metal, metal alloy, or a polymeric material.

4. The sheet according to claim 1, wherein the plastic deformable and non-toxic material is chosen from the group consisting of stainless steel or an alloy thereof, aluminium or an alloy thereof, niobium or an alloy thereof, zirconium or an alloy thereof, tantalum or an alloy thereof, or titanium or an alloy thereof.

5. The sheet according to claim 4, wherein the plastic deformable and non-toxic material is chosen from titanium or an alloy thereof.

6. The sheet according to claim 1, wherein the matrix is stretchable in both a longitudinal and transversal direction of the sheet.

7. The sheet according to claim 1, wherein the holes are stretchable in both a longitudinal and transversal direction of the sheet.

8. The sheet according to claim 1, wherein the matrix and the holes are stretchable in both a longitudinal and transversal direction of the sheet.

9. The sheet according to claim 1, wherein the sheet is more stretchable in a longitudinal direction than in a transversal direction, or vice versa.

10. The sheet according to claim 1, wherein the holes are non-round.

11. The sheet according to claim 1, wherein the holes are curved or angular.

12. The sheet according to claim 1, wherein the holes create a repetitive pattern on the sheet.

13. The sheet according to claim 1, wherein the sheet also comprises at least one preformed fixation hole.
